(19) United States
(12) Patent Application Publication
(10) Pub. No.: US 2010/0049088 A1
(43) Pub. Date: Feb. 25, 2010

(54) Specimen container

(75) Inventors: George William Luckhardt, San Diego, CA (US); Esther En-Yu Luckhardt, San Diego, CA (US)

Correspondence Address: George W Luckhardt (Law Offices), PO Box 927951, San Diego, CA, 92192, US

(73) Assignee: IP Filepoint, LLC, A Delaware LLC

(21) Appl. No.: 12/229,699

(22) Filed: Aug. 25, 2008

(51) Int. Cl. A61B 5/00

(52) U.S. Cl. 600/573

(57) ABSTRACT

A specimen container includes an opening for receiving biological fluid specimens, and a body, wherein at least one of a body mid-section and a container base is substantially wider than the opening. The body mid-section may be curved and wider than the container base. Alternatively, the body mid-section may be flat and narrower than the container base. A detachable lid may be configured to cover the opening. In another embodiment, a specimen container includes a base member, a set of wall members fixedly connected to the base member, and a deflector cap configured to deflect fluid specimen material back into the specimen container. Advantages of the present invention include a specimen container that allows more complete and easier specimen collection and retention. These advantages may be achieved, at least in part, through better shape optimization of the specimen container.

### FIELD

The invention relates generally to a specimen container and, more specifically, to a fluid specimen container.

### BACKGROUND

A fluid specimen container provides a receptacle for collection of biological fluids, such as blood, urine, or semen. The specimen container is used for collecting the specimen then holding it, such as while the specimen is delivered to a lab for analysis. Most fluid specimen containers are cylindrical or tapered cylindrical in shape, with the broad end of the taper being at the container opening (see, for example, U.S. Pat. Nos. 3,140,007; 4,300,404; and D516,223).

Therefore, a specimen container with better shape optimization to allow better specimen collection and retention is needed.

### SUMMARY

Accordingly, the present invention is directed to a specimen container that overcomes one or more problems due to limitations and disadvantages of the related art.

In one embodiment, a specimen container includes an opening for receiving biological fluid specimens, and a body, wherein at least one of a body mid-section and a container base is substantially wider than the opening. The body mid-section may be curved and wider than the container base. The body mid-section may be roughly spherical. Alternatively, the body mid-section may be flat and narrower than the container base. The overall shape of the body may be roughly conical. A detachable lid may be configured to cover the opening. The specimen container may be made of at least one of the group consisting of a plastic, a polymer, a synthetic rubber, a composite, a metal, and a ceramic.

In another embodiment, a specimen container includes an opening for receiving biological fluid specimens, and a body, wherein at least a portion of a body mid-section is situated substantially below the opening and a container base is situated substantially below the body mid-section, when the specimen container is in a substantially upright orientation. The overall shape of the body may be roughly a tear-drop shape. A detachable lid may be configured to cover the opening. The specimen container may be made of at least one of the group consisting of a plastic, a polymer, a synthetic rubber, a composite, a metal, and a ceramic.

In yet another embodiment, a specimen container includes a base member, a set of wall members fixedly connected to the base member, and a deflector cap, hingedly connected to a top of at least one wall member, the deflector cap configured to deflect fluid specimen material back into the specimen container. The shape of the base member may be selected from the group consisting of a square, a rectangular, a triangle, a curved-end rectangle, and a hemisphere. The deflector cap may be hingedly connected to the top of the at least one wall member by at least one flexible connection to the at least one wall member. The specimen container may be made of at least one of the group consisting of a plastic, a polymer, a synthetic rubber, a composite, a metal, and a ceramic. The specimen container may also include wall flaps, attached to at least one of the deflector cap and the wall members, the wall flaps configured to extend at least one wall member as the deflector cap is raised and to deflect the fluid specimen material back inside the specimen container. Each wall flap may be fixedly connected to the deflector cap.

Advantages of the present invention include a specimen container that allows more complete specimen collection. Another advantage of the present invention includes a specimen container that allows easier specimen collection. Another advantage of the present invention includes a specimen container that allows better specimen retention. These advantages may be achieved, at least in part, through better shape optimization of the specimen container.

### BRIEF DESCRIPTION OF THE INVENTION

The accompanying drawings, which are included to provide a further understanding of the invention and are incorporated in and constitute a part of this application, illustrate embodiments of the invention and together with the description serve to explain the principles of the invention.

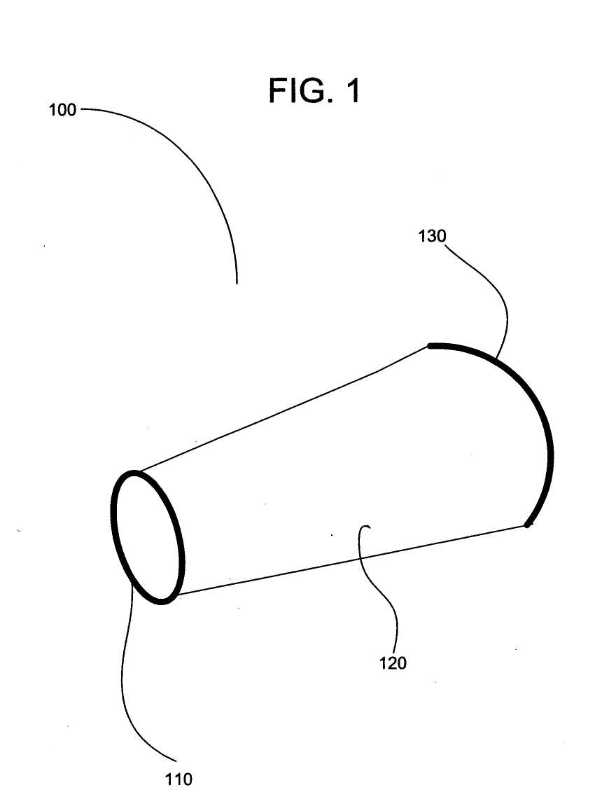

FIG. 1 is a diagram illustrating an exemplary specimen container, according to one embodiment of the invention.

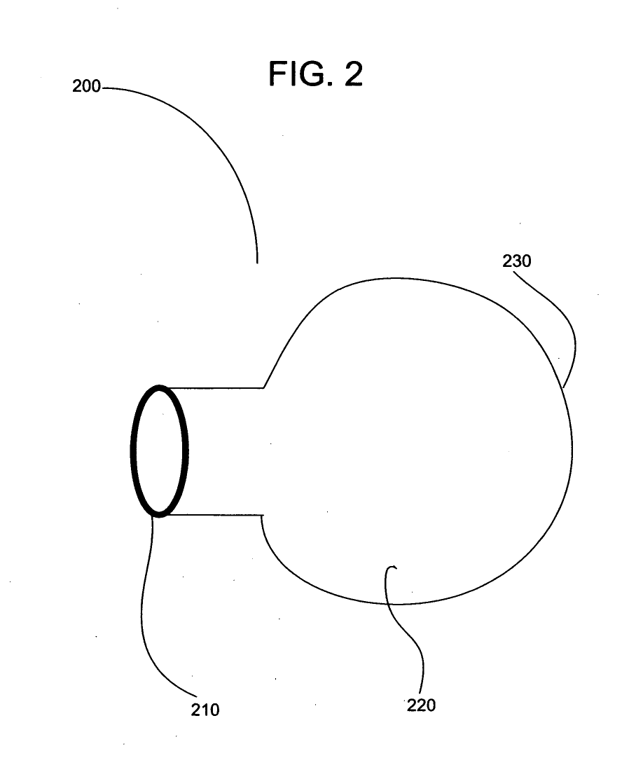

FIG. 2 is a diagram illustrating an exemplary specimen container, according to another embodiment of the invention.

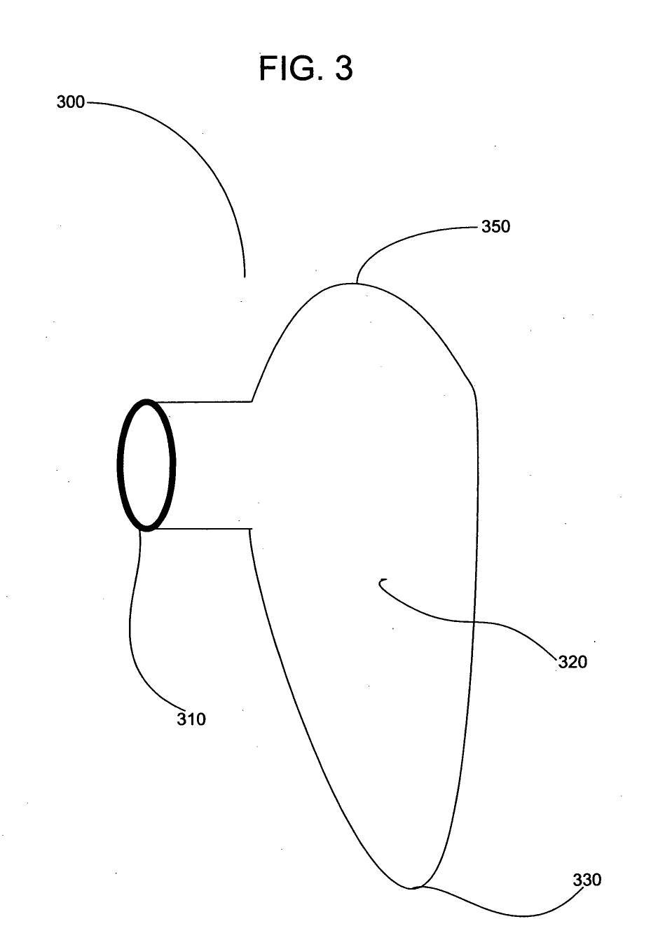

FIG. 3 is a diagram illustrating an exemplary specimen container, according to yet another embodiment of the invention.

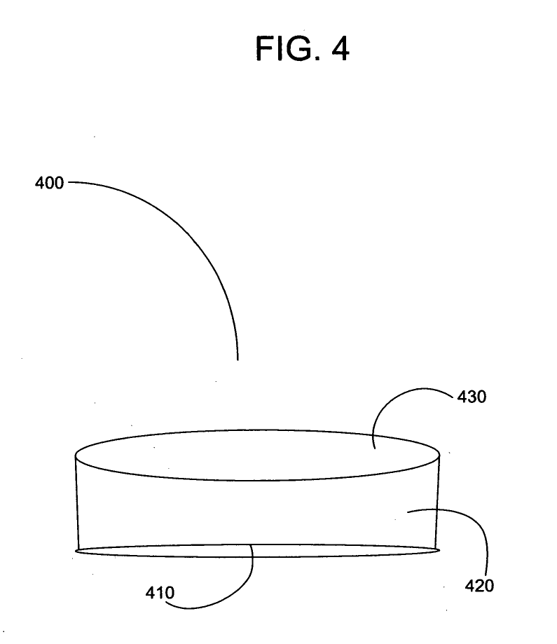

FIG. 4 is a diagram illustrating an exemplary container lid for use in conjunction with the exemplary specimen containers depicted in FIGS. 1-3.

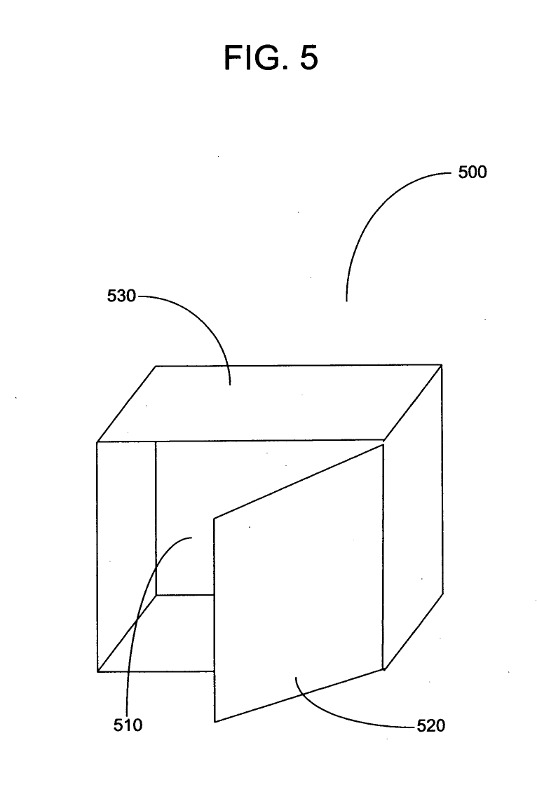

FIG. 5 is a diagram illustrating an exemplary specimen container, according to still another embodiment of the invention.

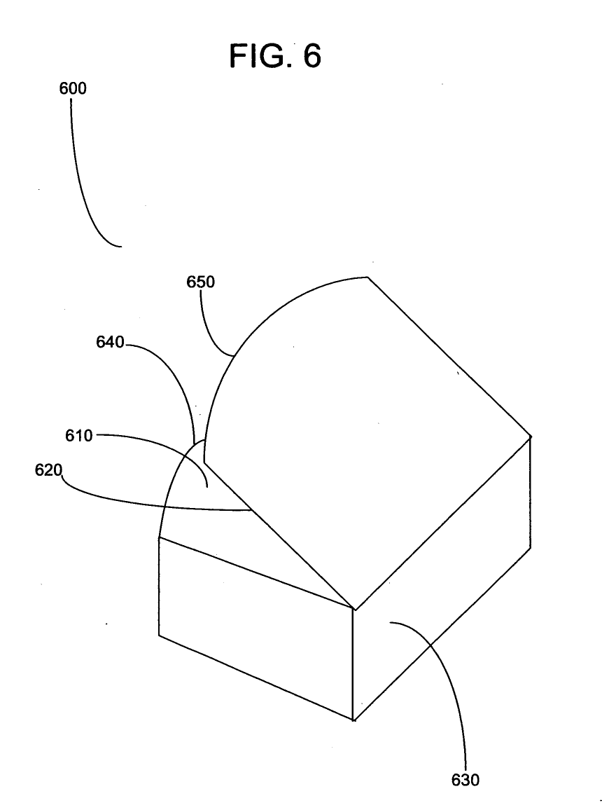

FIG. 6 is a diagram illustrating an exemplary specimen container, according to an embodiment of the invention that is a variation in shape from the exemplary specimen container depicted in FIG. 5.

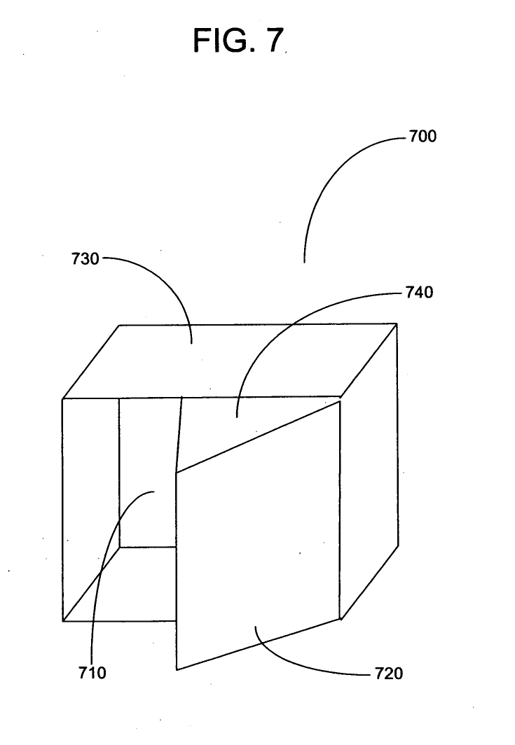

FIG. 7 is a diagram illustrating an exemplary specimen container having side flaps, according to an embodiment of the invention.

### DETAILED DESCRIPTION OF THE INVENTION

Reference will now be made in detail to the preferred embodiments of the present invention, examples of which are illustrated in the accompanying drawings. Wherever possible, the same reference numbers will be used throughout the drawings to refer to the same or like parts.

FIG. 1 is a diagram illustrating an exemplary specimen container, according to one embodiment of the invention. The specimen container 100 includes an opening 110, through which the specimen is received for collection, a container body 120 (e.g., body portion) for holding and containing the specimen, and a container base 130, to act as the supporting base of the specimen container. Note that the walls of the container body 120 taper outwardly from the opening 110 to the container base 130. Thus, the opening 110 has a smaller diameter than the container base 130. This conical shape with the narrow opening 110 may assist with specimen retention, as well as collection.

FIG. 2 is a diagram illustrating an exemplary specimen container, according to another embodiment of the invention. The specimen container 200 includes an opening 210, through which the specimen is received for collection, a container body 220 for holding and containing the specimen, and a container base 230, to act as the supporting base of the specimen container. Note that the walls of the container body 220, especially at the container body mid-section are substantially wider than the opening 210. Thus, the opening 210 has a smaller diameter than the container body 220. This spherical or pear-shaped container body 220, coupled with the narrow opening 210 may assist with specimen retention, as well as collection.

FIG. 3 is a diagram illustrating an exemplary specimen container, according to yet another embodiment of the invention. The specimen container 300 includes an opening 310, through which the specimen is received for collection, a container body 320 for holding and containing the specimen, and a container bottom 330 and a container top 350. Note that the opening 310 is narrower than the container body 320. Also note that the container body roughly has an inverted tear-drop shape, to allow for ease of use, collection and retention, with the opening 310 roughly perpendicular to the container body 320. This teardrop shaped or inverted tear-drop shaped container body 320, coupled with the narrow, perpendicular opening 310 may assist with specimen retention, as well as collection.

FIG. 4 is a diagram illustrating an exemplary container lid for use in conjunction with the exemplary specimen containers of FIGS. 1-3. The container lid 400 includes a lid top 430, a lid side 420, and a lid bottom opening 410. The lid side 420 may be used to twist the container lid 400 onto the specimen container at the lid bottom opening 410, while the lid top 430 provides covering on the top side of the lid/container. The lid bottom opening may employ a screw top, a snap top, or a click top, for example.

FIG. 5 is a diagram illustrating an exemplary specimen container, according to still another embodiment of the invention. The exemplary specimen container 500 is roughly in the shape of a rectangular box with a lid, and has a base member 510, a set of wall members 530 fixedly connected to the base member 510, and a deflector cap 520, hingedly connected to a top of at least one wall member, the deflector cap 520 configured to deflect fluid specimen material back into the specimen container. This roughly flat-shaped container 500 with deflector cap 520 may assist with specimen retention, as well as collection. It should be noted that, instead of a rectangular shape, the base member 510 may have a square, triangular, curved-end rectangle, or a hemispherical shape, for example.

FIG. 6 is a diagram illustrating an exemplary specimen container, according to an embodiment of the invention that is a variation in shape from the exemplary specimen container illustrated in FIG. 5. The exemplary specimen container 500 is roughly in the shape of a curved-end rectangular box with a lid, and has a base member 610, a set of wall members 630 fixedly connected to the base member 610, and a deflector cap 620, hingedly connected to a top of at least one wall member, the deflector cap 620 configured to deflect fluid specimen material back into the specimen container. The specimen container 600 also front edges of the curved-end of the rectangle, a base front member 640 and a cap front member 650. This roughly flat-shaped container 600 with deflector cap 620 may assist with specimen retention, as well as collection.

FIG. 7 is a diagram illustrating an exemplary specimen container having side flaps, according to an embodiment of the invention. The exemplary specimen container 700 is roughly in the shape of a rectangular box with a lid, such as specimen container 500, and has a base member 710, a set of wall members 730 fixedly connected to the base member 710, and a deflector cap 720, hingedly connected to a top of at least one wall member, the deflector cap 720 configured to deflect fluid specimen material back into the specimen container. The exemplary specimen container also has a set of wall flaps 740, attached to at least one of the deflector cap and the wall members. The wall flaps 740 serve to deflect fluid specimen material back inside the specimen container. This roughly flat-shaped container 700 with deflector cap 720 and the wall flaps 740 may assist with specimen retention, as well as collection

In one embodiment, a specimen container includes an opening for receiving biological fluid specimens, and a body, wherein at least one of a body mid-section and a container base is substantially wider than the opening. The body mid-section may be curved and wider than the container base. The body mid-section may be roughly spherical. Alternatively, the body mid-section may be flat and narrower than the container base. The overall shape of the body may be roughly conical. A detachable lid may be configured to cover the opening. The specimen container may be made of at least one of the group consisting of a plastic, a polymer, a synthetic rubber, a composite, a metal, and a ceramic.

In another embodiment, a specimen container includes an opening for receiving biological fluid specimens, and a body, wherein at least a portion of a body mid-section is situated substantially below the opening and a container base is situated substantially below the body mid-section, when the specimen container is in a substantially upright orientation. The overall shape of the body may be roughly a tear-drop shape. A detachable lid may be configured to cover the opening. The specimen container may be made of at least one of the group consisting of a plastic, a polymer, a synthetic rubber, a composite, a metal, and a ceramic.

In yet another embodiment, a specimen container includes a base member, a set of wall members fixedly connected to the base member, and a deflector cap, hingedly connected to a top of at least one wall member, the deflector cap configured to deflect fluid specimen material back into the specimen container. The shape of the base member may be selected from the group consisting of a square, a rectangular, a triangle, a curved-end rectangle, and a hemisphere. The deflector cap may be hingedly connected to the top of the at least one wall member by at least one flexible connection to the at least one wall member. The specimen container may be made of at least one of the group consisting of a plastic, a polymer, a synthetic rubber, a composite, a metal, and a ceramic. The specimen container may also include wall flaps, attached to at least one of the deflector cap and the wall members, the wall flaps configured to extend at least one wall member as the deflector cap is raised and to deflect the fluid specimen material back inside the specimen container. Each wall flap may be fixedly connected to the deflector cap.

Advantages of the present invention include a specimen container that allows more complete specimen collection. Another advantage of the present invention includes a specimen container that allows easier specimen collection. Another advantage of the present invention includes a specimen container that allows better specimen retention. These advantages may be achieved, at least in part, through better shape optimization of the specimen container.

It will be apparent to those skilled in the art that various modifications and variations may be made in the present invention without departing from the spirit or scope of the inventions. Thus, it is intended that the present invention covers the modifications and variations of this invention provided they come within the scope of the appended claims and their equivalents.

1. A specimen container, comprising:
an opening for receiving biological fluid specimens; and
a body, wherein at least one of a body mid-section and a container base is substantially wider than the opening.

2. The specimen container of claim 1, wherein the body mid-section is curved and wider than the container base.

3. The specimen container of claim 2, wherein the body mid-section is roughly spherical.

4. The specimen container of claim 1, wherein the body mid-section is flat and narrower than the container base.

5. The specimen container of claim 4, wherein the overall shape of the body is roughly conical.

6. The specimen container of claim 1, further comprising a detachable lid configured to cover the opening.

7. The specimen container of claim 1, wherein the specimen container is made of at least one of the group consisting of:
a plastic;
a polymer;
a synthetic rubber;
a composite;
a metal; and
a ceramic.

8. A specimen container, comprising:
an opening for receiving biological fluid specimens; and
a body, wherein at least a portion of a body mid-section is situated substantially below the opening and a container base is situated substantially below the body mid-section, when the specimen container is in a substantially upright orientation.

9. The specimen container of claim 8, wherein the overall shape of the body is roughly a tear-drop shape.

10. The specimen container of claim 8, further comprising a detachable lid configured to cover the opening.

11. The specimen container of claim 8, wherein the specimen container is made of at least one of the group consisting of:
a plastic;
a polymer;
a synthetic rubber;
a composite;
a metal; and
a ceramic.

12. A specimen container, comprising:
a base member;
a set of wall members fixedly connected to the base member; and
a deflector cap, hingedly connected to a top of at least one wall member, the deflector cap configured to deflect fluid specimen material back into the specimen container.

13. The specimen container of claim 12, wherein the shape of the base member is selected from the group consisting of:
a square;
a rectangular;
a triangle;
a curved-end rectangle; and
a hemisphere.

14. The specimen container of claim 12, wherein the deflector cap is hingedly connected to the top of the at least one wall member by at least one flexible connection to the at least one wall member.

15. The specimen container of claim 12, wherein the specimen container is made of at least one of the group consisting of:
a plastic;
a polymer;
a synthetic rubber;
a composite;
a metal; and
a ceramic.

16. The specimen container of claim 12, further comprising wall flaps, attached to at least one of the deflector cap and the wall members, the wall flaps configured to extend at least one wall member as the deflector cap is raised and to deflect the fluid specimen material back inside the specimen container.

17. The specimen container of claim 16, wherein each wall flap is fixedly connected to the deflector cap.
